(19) United States
(12) Patent Application Publication
(10) Pub. No.: US 2020/0230340 A1
(43) Pub. Date: Jul. 23, 2020

(54) RIGID STYLET APPARATUS WITH OPTIONAL SUCTION

(71) Applicants: John Alexander Cheyne, Vancouver (CA); Gillian Patricia Edworthy, Vancouver (CA)

(72) Inventors: John Alexander Cheyne, Vancouver (CA); Gillian Patricia Edworthy, Vancouver (CA)

(21) Appl. No.: 16/253,199

(22) Filed: Jan. 21, 2019

(51) Int. Cl. A61M 16/04

(52) U.S. Cl. CPC A61M 16/0463; A61M 16/0488

(57) ABSTRACT

A rigid stylet apparatus having a stylet body having a suction port, the rigid stylet port and an optional suction port. The stylet body has an internal passageway in fluid communication with the suction port, the rigid stylet port and the optional suction port. The apparatus further having a rigid stylet in fluid communication with the rigid stylet port. The optional suction port may have a thumb rest having an optional suction opening being in fluid communication with internal passageway. The thumb rest has a concave surface to allow for ease of use by an operator.

### REFERENCE TO PENDING APPLICATIONS

This application does not claim the benefit of any issued U.S. patent or pending application.

### TECHNICAL FIELD

The present invention relates generally toward a rigid stylet for use with endotracheal intubation, and more specifically, to a rigid stylet with optional suction capability.

### BACKGROUND

Endotracheal intubation, usually referred to as intubation, is the placement of an endotracheal tube (ETT), which is typically a flexible plastic tube, into the trachea to maintain and secure an open airway and to serve as a conduit to administer oxygen and in occasion, certain medications. Intubation is normally facilitated by using a conventional laryngoscope to provide the operator with a view of the vocal cords to which the ETT will pass through.

The ETT is placed into the trachea through the use of an intubating stylet. The stylet is a metal wire with primary function to allow the user to bend the ETT to the optimal shape of the individual patient anatomy. The ETT shape should be maintained during intubation in its desired shape, as determined by the operators manipulation of the stylet and therefore the ETT itself. There are multiple angles in which the ETT must go around, thus having a rigid stylet is critical in maneuvering the tube around the variable angles. A ETT without a stylet is flexible and can easily be caught up in any of these obstructive angles preventing a successful intubation. While there are different types of stylets, a rigid malleable stylet is the standard instrument used in intubation.

Viewing the vocal cords can be a critical challenge in ETT intubation. Increased secretions, vomit, and blood are the main fluid types preventing the view of the vocal cords. This fluid needs to be removed in order to view the vocal cords and pass the ETT through the vocal cords to secure the patients airway. ETT intubation generally requires a medically induced paralysis of the patient that prevents their cough reflex and swallowing capacity, resulting in the patients inability to clear these secretions. There are multiple medical conditions that cause significant increased airway swelling (ie. burns and toxidromes), blood in the airway or esophagus (gastrointestinal bleeds and pulmonary hemorrhages) or vomit/fecal matter in the airway. Secretions, blood or vomit can interfere with the operator's view of the vocal cords, resulting in a loss of oxygen to the patient. The longer that the foreign fluid remains in the airway, the greater the risk of harm to the patient. This fluid will inevitably enter the trachea and into the lungs resulting in hypoxia. Also, this fluid can lead to infection or chemical pneumonitis as the lungs fill with this fluid causing more inflammation in the lungs and a significant increases in morbidity and mortality. The Endotracheal tube (ETT) suction is therefore necessary to clear secretions and to maintain airway patency, and to optimize oxygenation and ventilation in a ventilated patient. ETT suction is typically conducted through the use of a separate suction probe.

A challenge is the typical patients airway can only accommodate two out of the three aforementioned devices needed to intubate due to the limited size of the airway, i.e. ETT, laryngoscope and suction probe, at any given time. As such, when secretions are in the airway of a patient being intubated, the endotracheal tube must be removed to make room for the suction probe as the laryngoscope always remains in the airway. When the suction probe is then removed, this allows the ETT to be reinserted for an additional attempt to secure the airway. During this extended time, the secretions may reoccur resulting in a repeat of the above. This can cause the patient to deoxygenate and decomensate. Further, any added hypoxic time can result in a significant increase in morbidity, even cardiorespiratory arrest and death.

U.S. Pat. No. 4,865,586 is a prior art attempt to resolve this challenge and is directed toward a suction guide for endotracheal intubation stylet. This device, however, provides continuous suction which results in fast deoxygenation. The device is a guide for the placement of an ETT rather then the stylet used for insertion, more specifically, this requires the ETT to be sleeved over the stylet after it has been passed through the cords. The estimated length of this patent for human use is longer then 3 feet.

U.S. Pat. No. 5,595,172 is another prior art attempt to resolve this challenge and is directed toward a suction stylet suited for use with an endotracheal tube. The two major differences are that this is not a rigid stylet, and the suction is not fully optional. This device is not designed with the use of a rigid stylet which is a critical part of the intubation process. Two design limitations of U.S. Pat. No. 5,595,172 results in oxygen continuously being removed from the patients airway. The mechanics of the optional vent port are located a significant distance from the vacuum attachment. Also, the tube radius of the two lumens, specifically the tube leading to the vent port, relative to the other tube radius of the flexible plastic tube within the ETT are approximatly 1:4. The result is a small amount of oxygen being continuously removed from the patients airway even when the optional vent port is not being initiated.

Accordingly, there is a need for a device to address the above challenges.

### SUMMARY

The present invention relates generally toward a rigid stylet for use with endotracheal intubation, and more specifically, to a rigid stylet with optional suction capability.

In one aspect, a rigid stylet apparatus is disclosed. This apparatus includes a stylet body having a suction port, the rigid stylet port and an optional suction port. The stylet body has an internal passageway in fluid communication with the suction port, the rigid stylet port and the optional suction port. The apparatus further includes a rigid stylet in fluid communication with the rigid stylet port.

In some aspects, the suction port and the rigid stylet port are inline while the optional suction port is substantially perpendicular to the suction port and the rigid stylet port. In other aspects, the optional suction port and the rigid stylet port are inline with the suction port being substantially perpendicular to the optional suction port and the rigid stylet port.

In some aspects, the optional suction port includes a thumb rest having an optional suction opening in fluid communication with internal passageway. The thumb rest has a concave surface to allow for ease of placement of the operator's thumb.

Other aspects and features of the present invention will become apparent to those ordinarily skilled in the art upon review of the following description of specific embodiments of the invention in conjunction with the accompanying figures.

### BRIEF DESCRIPTION OF THE DRAWING

In drawings which illustrate embodiments of the invention wherein similar characters of reference denote corresponding parts in each view,

FIG. 1 is a top perspective view of an embodiment of an apparatus of the present invention.

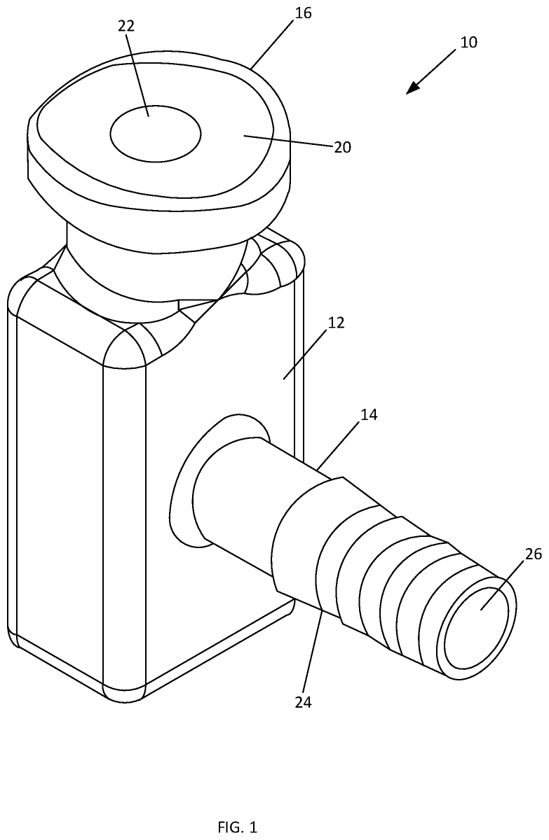

FIG. 2 is a bottom perspective view of an embodiment of an apparatus of the present invention.

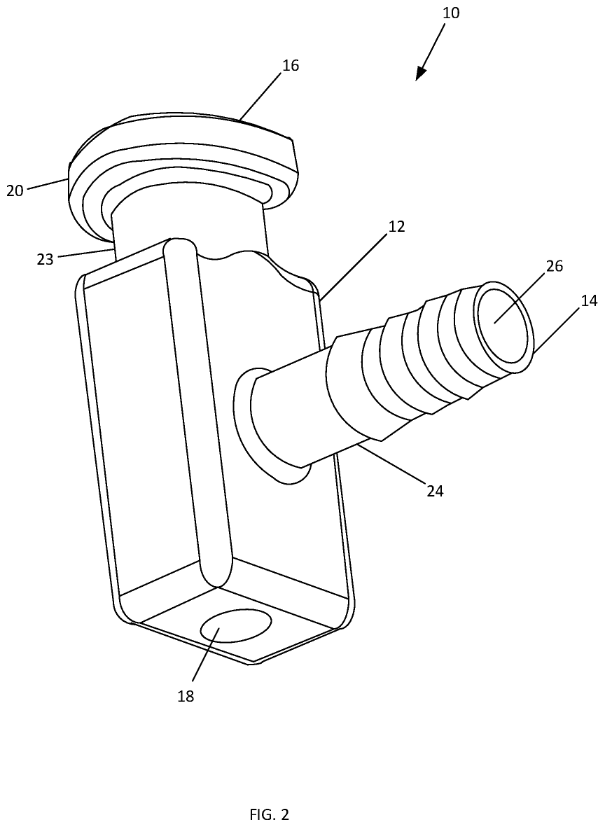

FIG. 3 is a side view of an embodiment of an apparatus of the present invention.

FIG. 4 is a top view of an embodiment of an apparatus of the present invention.

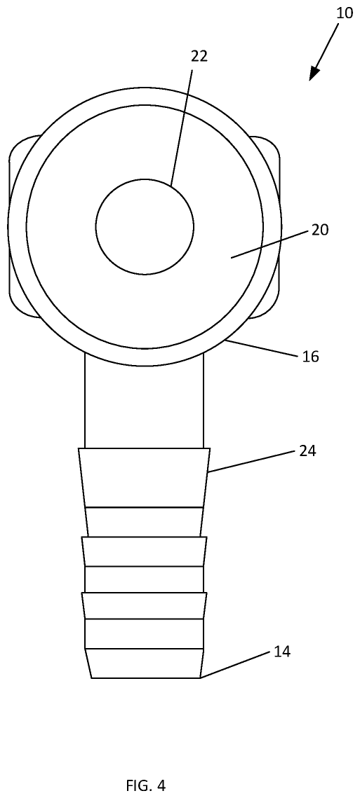

FIG. 5 is a cross-sectional view of an embodiment of an apparatus of the present invention.

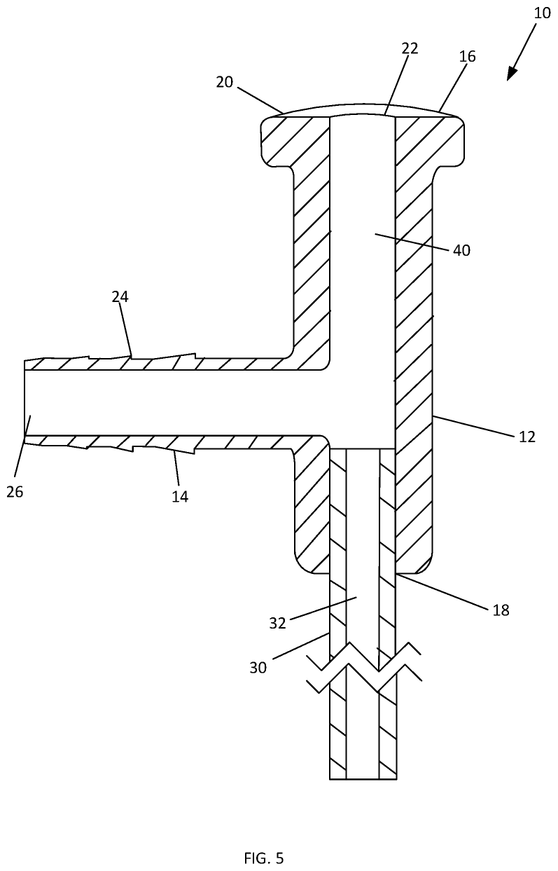

### DETAILED DESCRIPTION OF THE INVENTION

As illustrated in the Figures, an embodiment of a rigid stylet apparatus 10 of the present invention is disclosed. Apparatus 10 includes a stylet body 12 having a rigid stylet port 18, a suction port 14 and an optional suction port 16. Stylet body 12 further includes an internal passageway 40 which is in fluid communication with suction port 14, rigid stylet port 18 and optional suction port 16.

Apparatus 10 further includes a rigid stylet 30 which is dimensioned to be inserted into rigid stylet port 18. Rigid stylet 30 includes a stylet passageway 32 which allows for fluid communication with internal passageway 40. As apparatus 10 is designed for use with a rigid stylet 30, it is capable of handling and being utilized in all intubation situations which is not available in the prior art.

Rigid stylet 30 is designed to be placed inside an endotracheal tube (ETT) to assist with the insertion of the ETT into a patient's trachea. Suction port 14 and optional suction port 16 are designed to provide suction to a patient's airway in order to remove harmful secretions.

In this embodiment, optional suction port 16 and rigid stylet port 18 are in line with each other, i.e. optional suction port 16 is on a top surface of stylet body 12 while rigid stylet port 18 is on a bottom surface of stylet body 12. Suction port 14 is configured to be approximately perpendicular to optional suction port 16 and rigid stylet port 18, i.e. suction port 14 extends from a side surface of stylet body 12. This configuration allows for the placement of an operator's thumb over the optional suction port 16 while gripping stylet body 12 during use. This configuration allows for an ease and simplicity of use. Those skilled in the art, however, will recognize that other configurations of apparatus 10 may be utilized and are within the scope of the present invention. Other configurations may include suction port 14 and rigid stylet port 18 are in line with each other while optional suction port 16 is configured to be approximately perpendicular to optional suction port 14 and rigid stylet port 18.

In this embodiment, as illustrated by the Figures, suction port 14 includes an elongated fitting 24 having an opening 26. Fitting 24 is designed to be secured to an external hose or other device but assist with the suctioning of the patient's throat during the intubation process. Fitting 24 may be a standard shape of fitting for tubing that connects to all wall vacuum systems to allow suction.

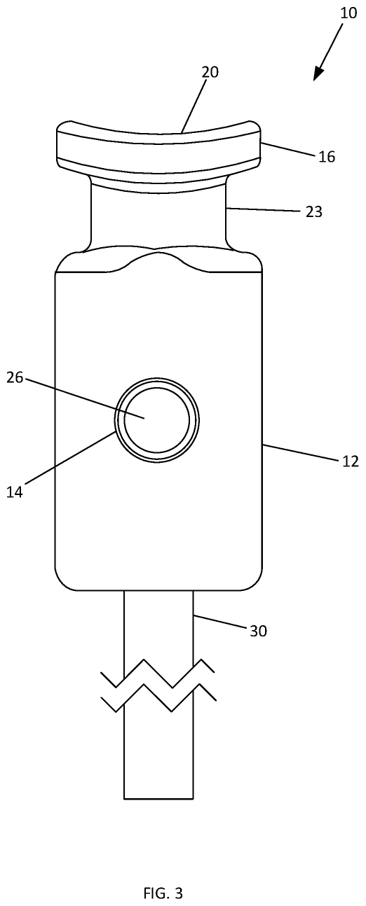

In this embodiment, optional suction port 16 includes a thumb rest 20 having an optional suction opening 22 which is in fluid communication with internal passageway 40. Thumb rest 20 is designed with a slight concave curvature as illustrated in FIG. 3. The slight concave curvature to allow for ease and accuracy of placement of the thumb during use. By having a slightly curved surface, the operator is more easily able to engage with optional suction opening 22 during use as the operator's thumb may be guided to optional suction opening 22 in the event of a slight misalignment of the operator's thumb during use.

In operation, suction port 14 is connect contact it to an external suction device which upon the closure of optional suction port 16 creates negative pressure within internal passageway 40 pulling airflow through rigid stylet passageway 32 and internal passageway 40 and exiting suction port 14. The allows for secretions and other items that could interfere with a patient's airflow, and operator's ability to intubate the patient, to be removed without the need for the removal of the ETT during the intubation process.

When optional suction port 16 is not closed, i.e. when it is opened, air flow is pulled through optional suction port 16 into internal passage way 40 and exiting suction port 14. This allows for intubation to occur without the need for suction.

By having the ability to optionally select when to engage suctioning through rigid stylet 30, the operate is given more control over the specific circumstances and conditions that are presented during intubation process. By having optional control. the capability of providing suction is readily available, while not providing constant suction which can lead to deoxygenation.

While preferred embodiments of the present inventive concept have been shown and disclosed herein, it will be obvious to those persons skilled in the art that such embodiments are presented by way of example only, and not as a limitation to the scope of the inventive concept. Variations, changes, and substitutions may occur or be suggested to those skilled in the art without departing from the intent, scope, and totality of this inventive concept. Such variations, changes, and substitutions may involve other features which are already known per se and which may be used instead of, in combination with, or in addition to features already disclosed herein. Accordingly, it is intended that this inventive concept be inclusive of such variations, changes, and substitutions, and by no means limited by the scope of the claims presented herein.

I claim:

1. A rigid stylet apparatus comprising:
a stylet body having a suction port, the rigid stylet port and an optional suction port, the stylet body having an internal passageway in fluid communication with the suction port, the rigid stylet port and the optional suction port; and
a rigid stylet in fluid communication with the rigid stylet port.

2. The rigid stylet apparatus of claim 1, wherein the suction port and the rigid stylet port being inline, and wherein the optional suction port being substantially perpendicular to the suction port and the rigid stylet port.

3. The rigid stylet apparatus of claim 1, wherein the optional suction port and the rigid stylet port being inline, and wherein the suction port being substantially perpendicular to the optional suction port and the rigid stylet port.

4. The rigid stylet apparatus of claim 1, wherein the optional suction port comprising:
a thumb rest having an optional suction opening, the thumb rest having a concave surface, the optional suction opening being in fluid communication with internal passageway.
